(19) United States
(12) Patent Application Publication
(10) Pub. No.: US 2006/0173071 A1
(43) Pub. Date: Aug. 3, 2006

(54) Mast cell stabilizers used to inhibit laminitis

(76) Inventor: Charles Foster Owen, Littleton, CO (US)

Correspondence Address: Charles F. Owen, P.O. Box 620694, Littleton, CO, 80162, US

(21) Appl. No.: 11/047,068

(22) Filed: Jan. 31, 2005

(51) Int. Cl. A61K 31/353

(52) U.S. Cl. 514/456

(57) ABSTRACT

The invention provides the use of mast cell stabilizers, which composition inhibits excess chemical release such as the mediator of laminitis, histamine, from mast cells for the prevention and/or treatment of laminitis. Mast cell stabilizers can be administered by way of inhalation, oral, subcutaneous, intramuscular or topical. Mast cell stabilizers may be administered before an onset of laminitis or administered during a laminitis attack to halt the stress response of laminitis.

### CROSS REFERENCES TO RELATED APPLICATIONS

### U.S. Patent Documents

U.S. Pat. No. 6,764,692
U.S. Pat. No. 6,534,526
U.S. Pat. No. 6,299,866
U.S. Pat. No. 6,283,219
U.S. Pat. No. 6,045,827
U.S. Pat. No. 5,891,472
U.S. Pat. No. 5,290,767
U.S. Pat. No. 5,204361

### BACKGROUND OF THE INVENTION

1. Field of the Invention

This invention relates to the prevention and/or treatment of laminitis. Laminitis is a debilitating hoof disease that can affect any hoofed animal, but primarily horses and cattle. Laminitis is defined as an inflammation of the sensitive laminae of the hoof, but also affects the body of the whole animal. The laminae is connective tissue that attaches the animals foot bone to the hoof wall, when this laminae becomes inflamed, it slowly dies and the bone and the hoof wall detach from each other which may cause a very devastating outcome.

2. Description of the Related Art

Many theories have been developed to explain how laminitis occurs. None of them, however mention Mast cell stabilizers; Histamine as the mediator of laminitis;

The trigger of laminitis is excess and/or difference from what the animals body can tolerate; Laminitis is a stress response. What they do mention is that laminitis has several causes such as pasture founder, grain overload, cushings disease, trauma founder and a lot of other causes which are being discovered every day. Theories of laminitis have ranged from cushings disease, thyroid disease, and bacteria forming agents that make the horse have laminitis along with theories relating to fructans in grass. In publications and patents such as:
U.S. Pat. Nos. 6,764,692; 6,534,526; 6,299,886; 6,283,219
U.S. Pat. Nos. 6,045,827; 5,891,472; 5,290,767; 5,204,361
   that state that there is mineral imbalances, or certain mechanism that describe the physiopathologic steps or pathways that tie to a common clinical lameness.

Researchers conclude that there is likely not one trigger factor or mechanism that initiates laminitis, but rather several pathways, which can switch on the disease. The current proposed etiology has different views; from reduced blood flow to the foot, increased blood flow to the foot, increased blood flow to the foot with shunting above the level of the sensitive laminae, basic inflammation of the foot, congestion of the digital vasculature and many others.

As work in the area of laminitis has progressed, multiple centers have developed methodologies and hypotheses as to the basic underlying cause of the disease. Data has been obtained and, the usual supporting and conflicting results from experimental work has been collected, published, and discussed.

Because of the complexity of the disease and the pathologic changes that occur relative to time during the developmental stage of laminitis, multiple hypotheses have been proposed to reconcile the experimental results to the endpoint of cellular death and support structure deterioration.

It should be noted that, with the passing of time, the remarkable advancement made in research tools for evaluating the normal homeostatic mechanism within the foot and the alterations to that homeostatsis continue to provide researchers better insight into the cascade of events resulting in clinical disease.

The goal of prevention is to inhibit the development of lameness. Optimal prevention requires a broad base approach ranging from altering management practices with the focus of decreasing exposure to know causes, to the timely use of specific drugs such as, x-adrenergic blocking agents (acetylpromazine, chlorpromazine, promazine); rheologic agents (pentoxifylline); antiplatelet agents (aspirin); COX 2 antagonists (phenylbutazone, flunixin meglumine); Oxygen radical scavengers (dimethyl sulfoxide(DMSO)); heparin; matrix metalloproteinase inhibitors (no drug available); and nitrous vasodilators (nitrous oxide). These drugs listed are sometimes in competition with each other, where one practitioner will use one type of drug and another practitioner will use another. There is not a consensus on what drug works the best and belief is that for each different cause of laminitis there is a drug that will be introduced to counteract the cause.

The goal of treatment of laminitis is focused on limiting the severity of the disease and enhancing the rate of recovery following the onset of lameness. Successful treatment will be reflected in a decrease in the number of horses that suffer mechanical failure of the foot.

In spite of the recent advancement in the theories of how laminitis develops along with how laminitis should be prevented and treated there is still not a conclusive singular pathway along with a single prevention or treatment that address the devastating disease of laminitis.

### SUMMARY OF BACKGROUND

The present invention involves a singular pathway along with a single prevention and/or treatment for laminitis. The singular pathway is precipitated by the stress response that is activated by a trigger of excess and/or difference from a stressor such as pasture founder, trauma founder, grain overload or from any other stressor. The single pathway from the stress response continues to the release of chemicals epinephrine and norepinephrine and together cortisol.

The chemicals epinephrine and norepinephrine become fatigued during the prolonged stress response and the cortisol misfires, thus signaling the body to release excess histamine to counteract the breakdown.

Excess histamine release is the mediator of laminitis which causes inflammation of the laminae where cell death occurs that may cause rotation of the foot bone.

As stated, excess histamine release is the mediator of laminitis. Histamine is released from mast cells in the animal's body during stress situations. If excess histamine is not released from mast cells then inflammation will not take place and therefore will not have laminitis. For prevention and/or treatment for laminitis and to counteract excess histamine release from mast cells, mast cell stabilizers are used.

Mast cell stabilizers inhibit the release of histamine and other chemicals from mast cells. Mast cell stabilizers that are used presently are Nedocromil Sodium, Cromolyn Sodium, Permirolast, Lodoxamise Trometamol, and Odoxamide, but new generation of mast cell stabilizers are being produced and improved and therefore would be under the umbrella of the present invention. Mast cell stabilizers can be administered by way of inhalation, oral, subcutaneous, intramuscular or topical. Mast cell stabilizers may be administered before an onset of laminitis or administered during a laminitis attack to halt laminitis and the stress response. Mast cell stabilizers are administered for the prevention and/or treatment on animals that are susceptible to laminitis such as horses, cattle, camels, goats, pigs, and sheep.

### DETAILED DESCRIPTION OF PREFERRED EMBODIMENTS

Before a person of ordinary skill in the art of the present invention of administering mast cell stabilizers for the prevention and/or treatment of laminitis, one needs to understand the laminitis process fully and grasp why mast cell stabilizers are effective and inhibiting laminitis and the stress response.

Laminitis is initiated by the stress response. The stress response is evoked by the trigger of laminitis which is excess and /or difference from what the animal's body can tolerate from the stressor. The stressor of laminitis is classified as any “cause” that can upset the homestasis of the animal's body if the animal gets too much of the stressor or a different stressor then what the animal's body can tolerate. Researchers use the word “cause” to recognize these stressors such as pasture founder, grain overload, trauma founder, road founder or any other stressor that upsets the homestasis of the animal. The stressor induces a signal, which travels to the central nervous system (CNS) through nervous and/or humoral pathways. The signal is received by the brain where the appropriate encoding and interpretation of the signal takes place. The first major axis or pathway, which is activated during the elicitation of the stress response is that of the autonomic nervous system via the hypothalamus.

This activation produces the “flight” or “fight” response and overall body arousal, which includes end-organ responses. During the “flight” or “fight” response, activity of the sympathetic nervous system is enhanced, while parasympathetic activity is decreased.

As a result of this sympathetic stimulation as well as increased secretion of epinephrine (a chemical that causes shunting of blood from the animal's extremities to the major organs) and norepinephrine (a chemical that causes vaso-constriction) by the adrenal medulla, the body is prepared for action.

The stress response is prolonged to the point that the chemicals epinephrine and norepinephrine become fatigued and they produce cortisol, which is an anti-inflammatory, but cortisol misfires under this prolonged stress. The body needs to counteract this breakdown so it releases histamine and other chemicals in excess amounts from mast cells making the blood system toxic. White blood cells are sent to kill of the excess histamine release and the other chemicals to bring the homestasis of the animal's body back to normal. By killing off the excess histamine by the white blood cells inflammation and the rest of the laminitis cascade is put in motion leading to ischemia and possible rotation of the foot bone.

If histamine and other chemicals are not released from the mast cells, then the white blood cells will not see a threat and inflammation will not take place, therefore there will not be laminitis. Histamine is the mediator of laminitis. To counteract excess histamine release mast cell stabilizers will be used.

The composition of mast cell stabilizers inhibit the release of excess histamine and other chemicals from mast cells and therefore stop laminitis from happening.

Mast cell stabilizers may be used before laminitis appears as a preventative medicine or it may be used for a treatment after laminitis has started. There are different mast cells stabilizers and new generations will be produce in the future, the best at the time of this present invention is Nedocromil Sodium. Nedocromil sodium is the disodium salt of 9-ethyl-6,9-dihydro-4,6-dioxo-10-propyl-4Hpyrano[3,2-g]quinoline-2,8-dicarboxylic acid. Nedocromil sodium is disclosed in British Patent Specification No: 2022078 and is useful in the prevention and/or treatment of laminitis. The patents that are relevant to this description of Nedocromil Sodium are:

| | | | |
|---|---|---|---|
| Patents: | 4,935,244; | 4,918,078; | 4,760,072 |

Nedocromil Sodium at the time of present invention is best administered through inhalation using a device with a spacer such as the product equine inhaler or any other type of animal inhaler that is on the market. It is without known side effects. It uses a metered dose inhaler. Recommendation is to research all animal inhalers and seek professional advice from manufactures of animal inhalers. Each animal with laminitis has different tolerances and resistance levels with different types of stressors.

The breed of the animal and past history also weighs in on the proper dosage for administering the mast cell stabilizer so that proper dosage should always be determined by a qualified veterinarian and never administered without the supervision of a veterinarian. Using Nedocromil Sodiumin in an un-safe manner will have consequences that may be detrimental to the animal.

What is claimed:

1. A method for prevention and/or treatment for laminitis in animals comprising the administering of mast cell stabilizers that inhibit histamine along with other chemicals from being released from mast cells.

2. A method as defined in claim 1, wherein said mast cell stabilizers comprising Nedocromil Sodium, Cromolyn Sodium, Pemirolast, Lodoxamise Trometamol and Odoxamide.

3. A method as defined in claim 1, wherein said mast cell stabilizer is administered by inhalation.

4. A method as defined in claim 1, wherein said mast cell stabilizer is administered orally.

5. A method as defined in claim 1, wherein said mast cell stabilizer is administered subcutaneously.

6. A method as defined in claim 1, wherein said mast cell stabilizer is administered intramuscularly.

7. A method as defined in claim 1, wherein said mast cell stabilizer is administered topically.

8. A method as defined in claim 1, wherein said animal is a horse.

9. A method as defined in claim 1, wherein said animal is selected from the group consisting of horses, cattle, camels, goats, pigs, and sheep.

10. A method as defined in claim 1, wherein said histamine is the mediator of laminitis.

11. A method as defined in claim 1, further comprising the trigger that initiates the seven sequences of the stress response of laminitis.

12. A method as defined in claim 11, wherein said trigger of laminitis is excess and/or difference from what the animal's body can tolerate.

13. A method as defined in claim 11, wherein said sequence number 1 is that the horse is exposed to a stressor, such as pasture founder.

14. A method as defined in claim 11, wherein said sequence number 2 is the trigger of laminitis, which is excess and/or difference from what the animal's body can tolerate.

15. A method as defined in claim 11, wherein said sequence number 3 is the stress response.

16. A method as defined in claim 11, wherein said sequence number 4 is auto-regulation or shunting of blood caused by the chemical epinephrine.

17. A method as defined in claim 11, wherein said sequence number 5 is vaso-constriction caused by the chemical norepinephrine.

18. A method as defined in claim 11, wherein said sequence number 6 is excess histamine release (vasodilation) caused by the misfire of cortisol from the adrenal gland.

19. A method as defined in claim 11, wherein said sequence number 7 is inflammation.

20. A method as defined in claim 11, wherein said laminitis is a stress response.
